(19) United States
(12) Patent Application Publication
(10) Pub. No.: US 2026/0004899 A1
(43) Pub. Date: Jan. 1, 2026

(54) SYSTEMS AND METHODS FOR DYNAMIC CONSENT BASED MANAGEMENT FOR MEDICAL INFORMATION SYSTEMS

(71) Applicant: Ethicon Endo-Surgery, Inc, Cincinnati, OH (US)

(72) Inventors: Manikandan Rajappa, North Wales, PA (US); Shantha Andrews, Lansdale, PA (US)

(21) Appl. No.: 18/757,719

(22) Filed: Jun. 28, 2024

(51) Int. Cl. G16H 10/60; G16H 40/20

(52) U.S. Cl. CPC G16H 10/60; G16H 40/20

(57) ABSTRACT

In examples, a method comprises validating a received patient credential and receiving, in response to success of the validation, one or more updated patient informed consent parameters. The method also comprises dynamically updating, in response to success of the validation, existing patient informed consent parameters based on the updated patient informed consent parameters. The updated patient informed consent parameters update: entity-access parameters to obtain an updated set of entities authorized to access a patient medical record, where the entities include organizations, one or more individual persons, groups of persons, or combinations thereof.

### TECHNICAL FIELD

This disclosure pertains to management of medical information systems and more specifically to dynamic informed consent based management for medical information systems.

### BACKGROUND

Various jurisdictions have enacted privacy, health, and other laws that constrain how certain patient medical information can be accessed or disseminated. In many instances, medical providers and other entities may not access, view, or distribute medical information unless a patient has provided informed consent. Informed consent refers to a voluntary agreement by an individual to participate in a particular activity or treatment and/or acquiesce to or authorize actions by others after receiving information relevant to decision-making. Informed consent may be based on legal and ethical guidelines and serve to promote a clear understanding of the risks, benefits, alternatives, and any other relevant information that is provided to patients.

### SUMMARY

In examples, a method comprises validating a received patient credential and receiving, in response to success of the validation, one or more updated patient informed consent parameters. The method also comprises dynamically updating, in response to success of the validation, existing patient informed consent parameters based on the updated patient informed consent parameters. The updated patient informed consent parameters update: entity-access parameters to obtain an updated set of entities authorized to access a patient medical record, where the entities include organizations, one or more individual persons, groups of persons, or combinations thereof.

In examples, a system comprises a memory, a communications interface, and a processor coupled to the communications interface. The processor is configured to receive and validate a patient credential of a patient. The processor is configured to, in response to success of the validation, receive one or more updated patient informed consent parameters. The processor is configured to dynamically update, in response to success of the validation, existing patient informed consent parameters based on the updated patient informed consent parameters, where the updated patient informed consent parameters update: entity-access parameters to obtain an updated set of entities authorized to access a patient medical record, where the entities include organizations, one or more individual persons, groups of persons, or combinations thereof.

In examples, a non-transitory, computer-readable medium stores executable instructions which, when executed by a processor, cause the processor to validate a received patient credential, and receive, in response to success of the validation, one or more updated patient informed consent parameters. The processor is also caused to dynamically update, in response to success of the validation, existing patient informed consent parameters based on the updated patient informed consent parameters, where the updated patient informed consent parameters update at least one of: a set of entities authorized to access a patient medical record, entity-specific access authorizations to specific portions of the patient medical record, and use-specific authorizations, the entities including one or more individual persons, groups of persons, or combinations thereof.

### BRIEF DESCRIPTION OF THE DRAWINGS

FIG. 1 is a block diagram of an example system for dynamically managing informed consent, in accordance with various examples.

FIG. 2 is a block diagram of an example system for dynamically managing informed consent, in accordance with various examples.

FIGS. 3-6 are examples of data structures associated with dynamic management of informed consent, in accordance with various examples.

FIG. 7 is a flow diagram of a method for dynamically managing informed consent, in accordance with various examples.

FIGS. 8-13 are graphical user interfaces for dynamically managing informed consent, in accordance with various examples.

### DETAILED DESCRIPTION

Many considerations including ethics, privacy, security, usage, and other laws, regulations, and practices may place conditions on the dissemination of patients' confidential medical information. Medical providers and other entities involved in medical care may face constraints when they attempt to access, use, or distribute a patient's medical information. The term entity refers to person(s) and/or organization(s) that may be eligible to request access to a patient's medical records. The term sub-entity refers to person(s) and/or group(s), department(s), etc. within an organization (“entity”). The constraints may depend on the nature and/or type of consent that the patient has provided. The term “informed consent,” as used herein, qualifies the consent provided by a patient granting access to an entity to indicate that the patient has been informed about various parameters associated with the access, use, distribution, etc. of an entity's request prior to obtaining consent. Conventionally, a patient may sign a blanket informed consent document that provides consent to all potential users (including clinicians (e.g., physicians, nurses, physician assistants, pharmacists) and non-clinicians (e.g., insurers, hospital administrators, medical researchers, medical technologists, medical transcriptionists)) of the patient's medical information, as part of the routine admission procedure in a clinical setting. For example, a patient visiting a hospital emergency room may be presented with an informed consent document which, when signed by the patient, enables the hospital to freely distribute the patient's medical information to any and all entities described in the document, which often includes all hospital employees. In many cases, the scope of conventional authorization in informed consent documents is unnecessarily broad and provides access to entities or users who have no specific reason to access the patient's medical information. Further, in conventional schemes, revoking informed consent that was granted earlier can be challenging. Typically, providing such consent to a narrower subset of entities than listed on informed consent documents may not be available to patients. In addition, depending on the location of the patient, patient data users, entities and/or providers, differing laws and regulations may apply. Accordingly, maintaining compliance with privacy, consent, and other laws applicable to the patient data across multiple jurisdictions may prove challenging and cumbersome. For example, some jurisdictions may enable patients to specify (e.g., opt-in or opt-out) use of their data accessed by healthcare professionals such as for clinical (e.g., primary) use or for other non-clinical (e.g., secondary) uses. Jurisdictions may also specify that patients may specify if their data is to be anonymized or pseudonymized prior to such non-clinical use where appropriate. In addition, jurisdictions may place additional patient consent measures and/or constraints on more sensitive data (e.g., genetic data) for research purposes. Accordingly, even from an institutional standpoint, the smorgasbord of measures governing consent can present substantial challenges to their ability to efficiently use patient information and involve substantial cost and resources. Moreover, in conventional approaches, patients are often reliant on entity (e.g., hospital or other institution) IT personnel or policies to ensure that their data is not accessed, used, and/or distributed by personnel or sub-entities without a valid purpose thereby potentially compromising patient privacy and the security and/or integrity of their data. In many instances, patients may even be unaware of entity policies and/or entity data protection measures.

Some disclosed embodiments pertain to systems and methods for the dynamic modification of access, use, and/or distribution controls for patient information. In some embodiments, the dynamic modification may be based on any existing patient informed consent, which may be modified dynamically by the patient. The term “dynamic informed consent” refers to the ability (e.g., by a patient) to tailor informed consent parameters at any point in time, for example, to: the entities/sub-entities to which the informed consent parameters apply, and/or the types of access, and/or specify use of the data by the entities/sub-entities, and/or tailor some informed consent parameters to specific types of patient data. Accordingly, the patient may: (a) apply access authorizations (e.g., granting, removing, or modifying access and/or distribution capabilities) represented by one or more informed consent parameters to a larger, smaller or different set of entities (entity-specific informed consent), and/or (b) change access authorizations to individuals, organizations, departments, etc., within those entities (sub-entity specific), and/or (c) change the nature of consent provided to each entity/sub-entity (e.g., use-specific—such as clinical, research, billing, insurance, etc., which may also occur on a per-entity/per-sub-entity basis), and/or (d) change the type of data to which the informed consent parameters apply for each entity/sub-entity (e.g., data-specific—such as genetic, procedure-related, disease/treatment related, etc.), and/or (e) specify actions (anonymization/pseudo-anonymization, encryption, security, etc.) to be taken prior to access and distribution, and/or (f) specify temporal, event-related, or other conditional parameters for access. Consent applicable to and/or tailored on a sub-entity basis may be referred to herein as “entity-access granularity.” Entity-access granularity may refer to the ability to specify: (a) one or more sub-entities, such as departments, personnel, sites, etc., that can access a patient medical record and (b) other sub-entities that may be prevented from accessing the patient medical record.

In particular, the examples described herein enable patients to dynamically customize the informed consent provided at various levels of granularity—in terms of access (which entities/sub-entities), usage (how the data can be used), data type (the type of data to which access and usage permissions apply—e.g., genetic, procedure-related, etc.), and temporal (e.g., how long the informed consent applies), which may be date-specific or event-specific (e.g., until the end of a procedure/treatment, billing completion, etc.) and/or measure-specific (e.g., measures to be taken anonymization, security, etc. prior to access, usage, and/or distribution). The examples described herein facilitate tailoring users to whom medical information is provided by enabling the patient to select, at a more granular level than previously possible, the specific users who may and who may not have access to that patient's medical information. In some embodiments, access to and/or distribution of patient information may be revoked or changed based on changes to informed consent parameters by the patient or by an authorized entity. By facilitating dynamic customization of informed consent with a high degree of precision, some disclosed embodiments enable the medical information system to more accurately capture and realize expressed patient privacy and to mitigate privacy, security, access, and usage concerns and limit the potential for unauthorized disclosure or exposure.

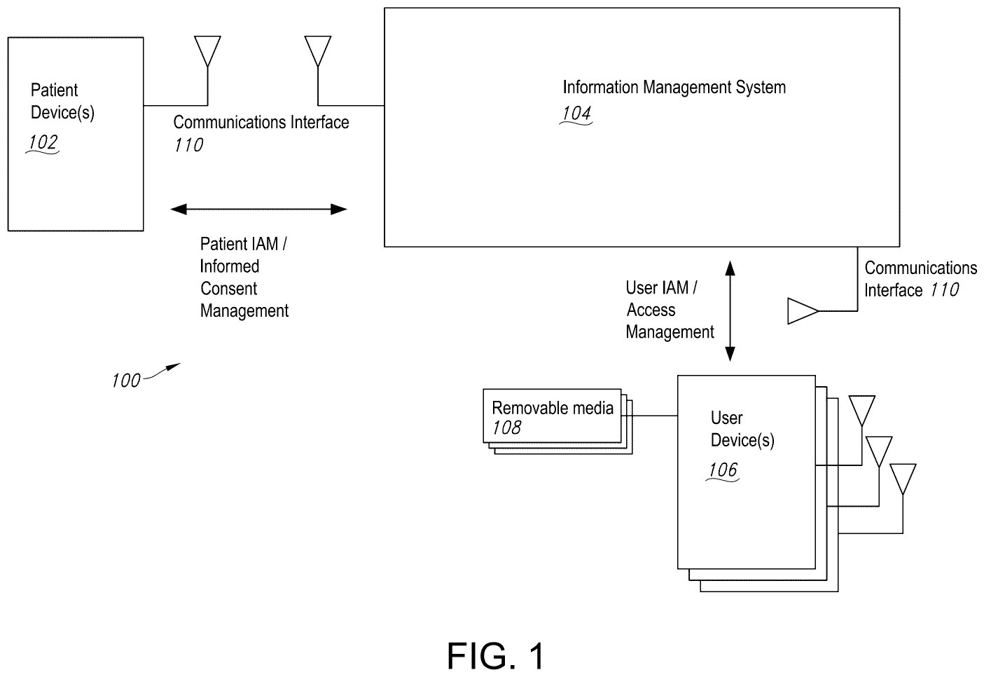

FIG. 1 is a block diagram of a system 100 for dynamically managing informed consents, in accordance with various examples. The system 100 may be located within a single building or on a single campus (e.g., a hospital building or university campus), or the system 100 may be distributed across multiple geographic locations (e.g., different components of the system 100 may be located in different buildings, campuses, cities, or countries). The system 100 may include patient device(s) 102, an information management system (IMS) 104, and one or more user devices 106. The patient device 102 may be any suitable electronic device with which a patient may dynamically manage the patient's informed consents. For example, the patient device 102 may include a smartphone, a tablet, a notebook, a laptop computer, a desktop computer, mobile devices such as cell phones, etc. The patient device 102 may also include and/or be coupled to an audio-based electronic component, such as a smart speaker or virtual assistant speaker. Such a speaker may be configured to authenticate the patient using capabilities provided by the patient device 102, such as by voice recognition, biometrics (e.g., fingerprint scanning, retinal scanning), and so on.

Prior to use, the patient may be associated with the patient device 102 by way of a registration process. During the registration process, the patient may provide the patient device 102 with various types of information, such as name, birthdate, social security number or other government-issued identification number, phone number, address, and so on. The patient device 102 may prompt the patient to provide authentication information, such as passwords, passcodes, biometric information including voice samples, and/or other samples (e.g., fingerprints, retinal scans), and so on. Other information may be solicited from the patient for patient identity, authentication, and access management (IAM) purposes. Once registration is complete, the patient may use the patient device 102 after logging in (e.g., using a local application, web-based service, etc.) as described herein. During use, communications between the patient device 102 and the IMS 104 may be handled using communications interface 110, which may be wired and/or wireless. Wired communications may include communications over Ethernet, Universal Serial Bus (USB), etc. Wireless communications may occur over: wireless wide area networks (WWANs) such as cellular networks, which may include LTE and/or 5G and/or other cellular communication standards; wireless local area networks (WLANs), e.g., Wi-Fi, which may be based on IEEE 802.11 standards, and/or wireless personal area networks (WPAN) such as Bluetooth, NFC, etc. which may be based on IEEE 802.15 standards. In general, communications between patient device(s) 102, user-device(s) 106, and IMS 104 may occur using some combination of the protocols outlined above. Further, in some embodiments, communications may be secured using various techniques, such as wired equivalent privacy (WEP), WiFi-protected access (WPA), WiFi-protected access II (WPA II), WiFi-protected access III (WPA III), advanced encryption standard (AES), etc.

The patient device 102 may be configured to communicate wired and/or wirelessly (e.g., via an Internet or other network connection) with other devices in the system 100, such as with the IMS 104 and/or with one or more of the user devices 106 using communications interface 110 on the patient device 102. The patient device 102 may belong to the patient or may be associated with the patient, such as during intake procedures in hospital admissions. The patient device 102 may include components that enable the patient device 102 to perform the various functions attributed herein to the patient device 102. For example, the patient device 102 may include a processor, a memory, communications interface, and storage (including removable media) coupled to the processor. The memory and/or storage may include code that the processor can execute to perform the functions attributed herein to the patient device 102.

The IMS 104 operates to process access and consent requests, and manage patient consent and modifications thereto. IMS 104 may include processors, memories, communications interfaces, and storage (including removable media) coupled to the processors. IMS 104 may operate as a cloud-based service (e.g., SaaS) and/or may leverage cloud-based resources. For example, the IMS 104 may store, and/or access information related to patient informed consent. The IMS 104 may dynamically manage consent including authenticating, receiving, adding, updating, deleting, and/or managing various modifications to a patient's informed consent from the patient device(s) 102, thereby enabling the patient to dynamically customize the patient's informed consent.

In some embodiments, the IMS 104 may also provide user IAM services to users/user device(s) 106. Upon gaining access, some user devices 106 (e.g., those devices into which a system administrator is logged in with appropriate security credentials) with patients' up-to-date informed consents when requested by the user device(s) 106. For other types of users (e.g., non-administrators), the IMS 104 may provide the user device(s) 106 with varying levels of detail regarding the patients' informed consents (e.g., in accordance with the type of consent, scope of consent, parties to the consent, entities authorized in the consent, sub-entities authorized in the consent, entities from whom consent is withheld, sub-entities from whom consent is withheld etc.), and in some examples, a user request may trigger a prompt to the patient(s) to authorize or deny the user request (such as the provision of some appropriate subset of the patient's informed consent records to the user). As described in detail below, the IMS 104 may include components that enable the IMS 104 to perform the various functions attributed herein to the IMS 104. For example, the IMS 104 may include a processor and a memory coupled to the processor. The memory (e.g., random access memory, dynamic random access memory, read only memory, non-volatile random access memory, flash memory, etc.) may store code that the processor can execute to perform the functions attributed herein to the IMS 104. In some embodiments, IMS 104 may be a server or network of servers, cloud-based infrastructure or service, and/or other networked computing device(s) capable of accommodating large volumes of network traffic and processing and storing large volumes of data.

The one or more user devices 106 may include smartphones, tablets, notebooks, laptop computers, desktop computers, mobile devices, etc. The user devices 106 may access the IMS 104 to request and/or obtain up-to-date patient informed consent. For example, in a clinical setting, IMS 104 or a patient care coordinator (e.g., in a situation where the requestor does not have access to IMS 104) may receive a request for a patient's medical information from a particular clinician. Before providing that clinician with the patient's medical information, IMS 104 or the patient care coordinator may access and obtain a patient's current informed consents. The informed consents may identify the entities (e.g., individual persons or groups) to whom the patient has provided informed consent and the entities to whom the patient has expressly refused informed consent. Based on this information, IMS 104 or the patient care coordinator may agree, refuse, or request patient authorization to provide the patient's medical information to the clinician requesting the information. In some embodiments, the one or more user devices 106 may include components that enable that user device 106 to perform the various functions attributed herein to the user device 106. For example, user devices 106 may include a processor, communications interface 110, a memory coupled to the processor, and storage (including removable storage media). The memory may store code that the processor can execute to perform the functions attributed herein to that user device 106. Further, in some embodiments, the one or more user devices 106 may be coupled to removable media 108, such as a USB device, a flash memory device, an optical memory device, a magnetic memory device, solid state memory devices, and so on.

In an example operation, the IMS 104 may store a patient's informed consent (e.g., in one or more data structures and/or databases, as described in the examples below). The patient may wish to dynamically customize the informed consent at some point in time. In some embodiments, the patient may use the patient device 102 to access the IMS 104. For example, the patient may enter a credential into a graphical user interface (GUI) displayed on the patient device 102, thereby granting the patient access to the patient's current informed consent (if any) and the ability to dynamically add and/or manage the informed consent as desired. The patient may view, add, or remove entities on the informed consent. The patient may customize the informed consent at a high level of granularity, for example, by granting informed consent to a group (e.g., a cardiology department in a hospital), but withholding consent from specific individuals within that group (e.g., a cardiologist in the cardiology department with whom the patient does not want patient medical information shared). The IMS 104 stores the patient's updates to the patient's informed consent. Subsequent attempts to access the patient's informed consent by way of the user device(s) 106 are served by providing the user device(s) 106 with patient medical records that are consistent with a current version of the patient's informed consent, denying the request (e.g., if the current version of the patient's informed consent indicates that the user may not access the patient's medical record), or sending an authorization request to the patient (to approve or deny the request).

The configuration of the system 100 shown in FIG. 1 is merely an example. Other configurations of the system 100 are contemplated and included in the scope of this disclosure.

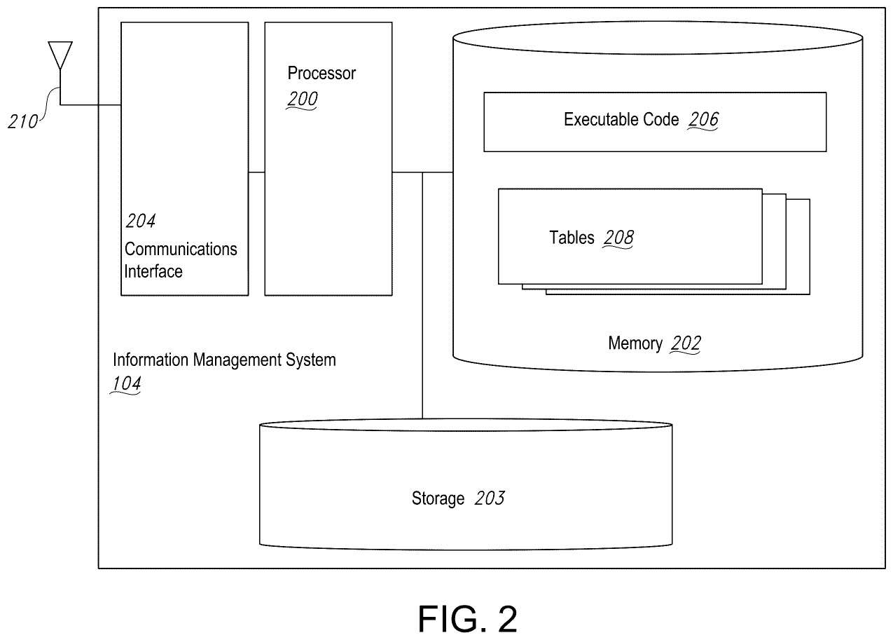

FIG. 2 is a block diagram of the IMS 104 to facilitate dynamic management of informed consent, in accordance with various examples. The IMS 104 may include one or more processors 200, memory 202, and storage 203 coupled to the processor 200. The IMS 104 may further include a communications interface 204 (e.g., a wired interface or a wireless interface) coupled to the processor 200. The memory 202 may store executable code 206 and tables 208 (e.g., data structures, databases, etc.). The executable code 206 may comprise a single or multiple instances of executable code, and the tables 208 may be included in single or multiple databases or data structures. The processor 200 executes the executable code 206 to perform the operations attributed herein to the IMS 104. The processor 200 may transmit and receive signals wirelessly via the communications interface 204 and an antenna coupled to the communications interface 204 (e.g., to communicate with the patient device 102 and/or the user device(s) 106 of FIG. 1). The processor 200 may include any suitable type of processor (e.g., single-core or multi-core processors, Application Specific Integrated Circuits (ASICs), etc.), and the memory 202 may include any of a variety of memory, such as random access memory (RAM), read-only memory (ROM), flash memory, etc. The storage 203 may include disks (e.g., optical or magnetic), solid state drives, hard disk drives, removable media (e.g., storage devices connected by USB), and so on. In some embodiments, the executable code 206 and/or the tables 208 may be stored in the storage 203 and transferred to the memory 202 for execution by the processor 200. The patient device(s) 102 and the IMS 104 may communicate by a communications interface that is wired (e.g., universal serial bus, ethernet, serial communication recommended standard 232) or wireless (e.g., wireless local area network (Institute of Electrical and Electronics Engineers (IEEE) 802.11), wireless wide area network (cellular, Long-Term Evolution (LTE)/5G), wireless personal area network (IEEE 802.15)).

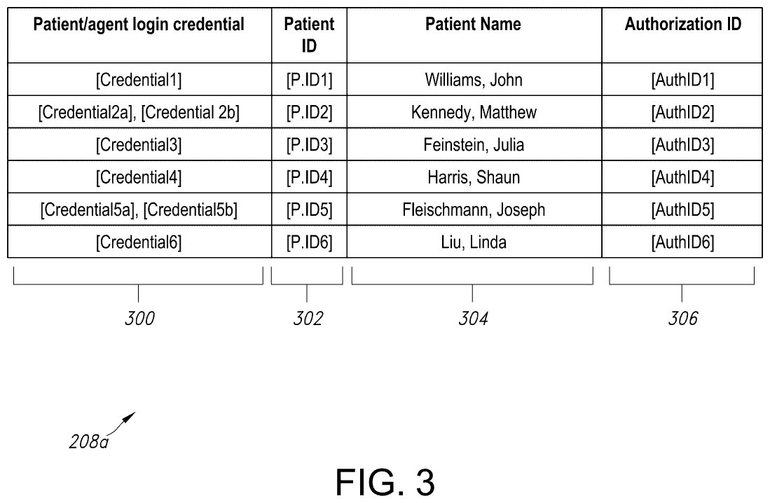

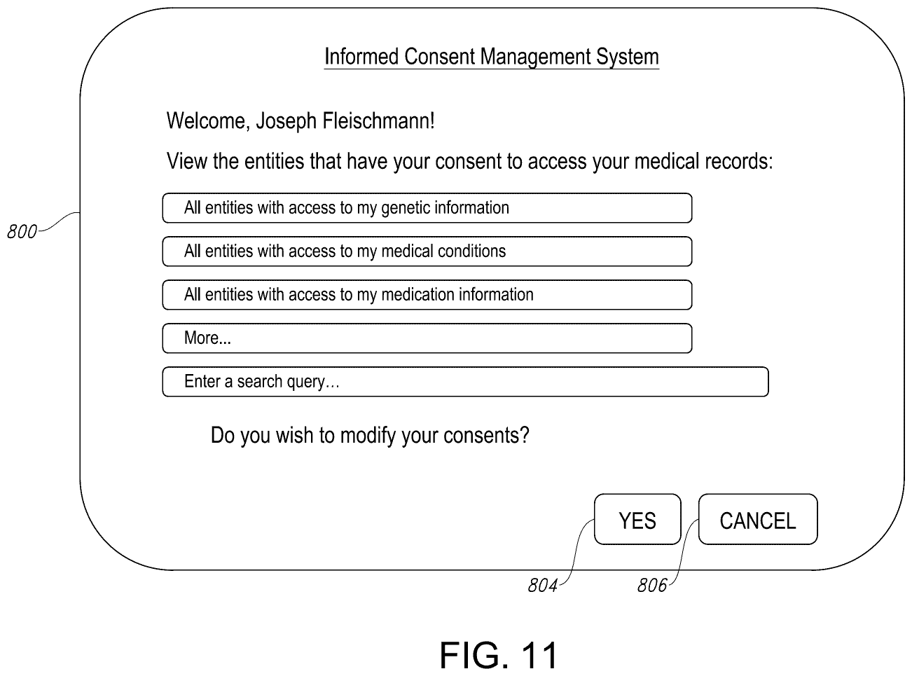

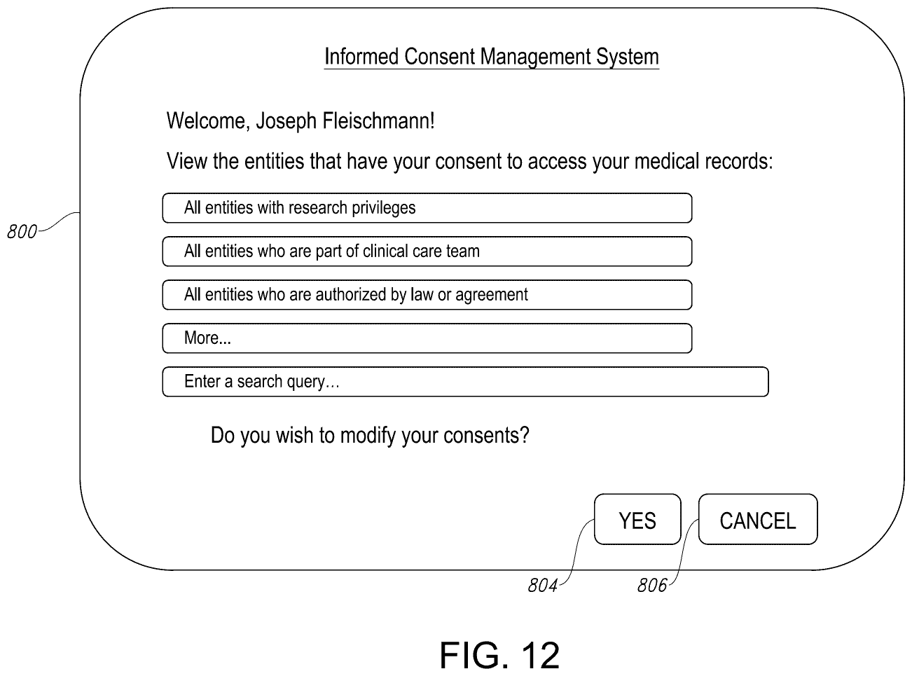

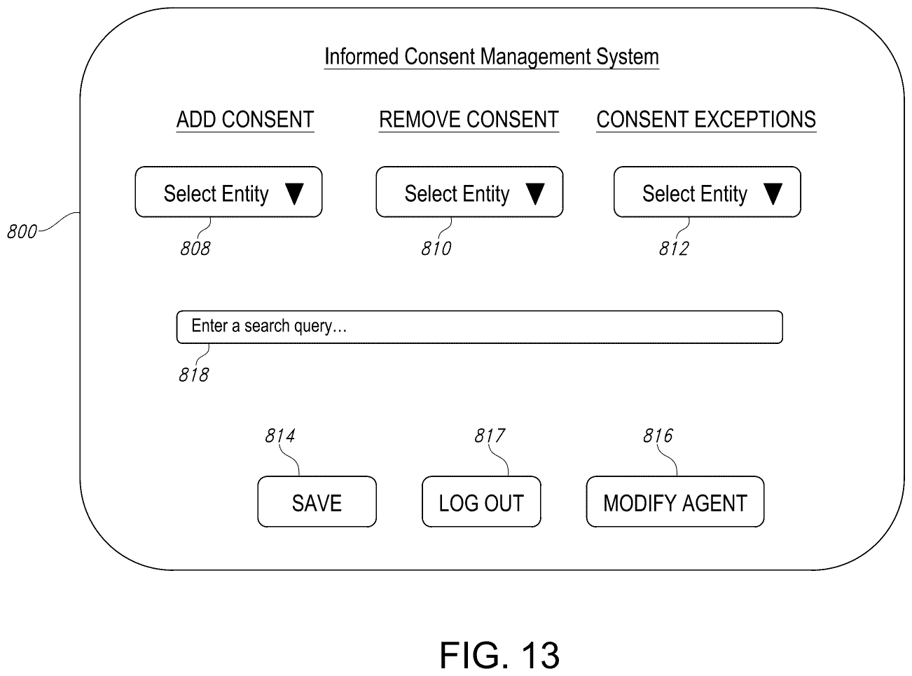

FIGS. 3-6 show example tables 208 to illustrate the operation of portions of system 100, such as in the IMS 104 of the system 100 of FIG. 1, for dynamically managing informed consents. The tables 208 of FIGS. 3-6 are depicted in a manner that facilitates understanding by the reader, but in practice, the tables 208 may be implemented differently than shown. In particular, FIG. 3 depicts an example table 208a including columns 300, 302, 304, and 306. Column 300 includes login credentials by which a patient (or a patient's authorized agent, such as a next-of-kin or a person holding valid verified legal authorization) may access and manage the patient's informed consents. Column 302 includes patient IDs, which can be alphanumeric or in any other appropriate encoding. Column 304 includes patient names corresponding to the respective patient IDs and login credentials, and column 306 includes references to each respective patient's informed consents. For example, patient Joseph Fleischmann is globally identified in the system 100 using patient ID [P.ID5]. The “P” signifies patient status, and the “ID” indicates that the value is an ID. Furthermore, the table 208a associates Mr. Fleischmann with login credentials [Credential5a] and [Credential5b]. (As with the patient ID and authorization ID, the actual login credentials may differ, but, as explained above, the example tables 208 and the example contents therein are formatted to facilitate reader understanding.) The [Credential5a] may belong to Mr. Fleischmann, while the [Credential5b] may belong to Mr. Fleischmann's next-of-kin, who also has access to Mr. Fleischmann's informed consents and is authorized to manage the informed consents. The table 208a associates Mr. Fleischmann with [AuthID5], which is associated with the specific informed consents provided by Mr. Fleischmann, as described below. The informed consents described herein may be searchable by entity, sub-entity, medical provider, scope of consent, type of consent, nature of consent (e.g., the search feature may identify which entities have been granted access to specific information, such as genetic information). For example, a user may use a device and a GUI displayed on the device, such as those described below with reference to FIG. 11 (search query box shown in example GUI 800), FIG. 12 (search query box shown in example GUI 800), and FIG. 13 (search query box 818), to specify a particular entity (e.g., Plainview Hospital), or a particular sub-entity (e.g., radiology department at Plainview Hospital), or a medical provider (e.g., Dr. Aaron Thomas in the radiology department at Plainview Hospital), or any other particular aspect such as those expressly listed above as well as other aspects not specifically enumerated herein, to search for and identify provided informed consents. Thus, for instance, searching for Plainview Hospital will return all informed consents provided to Plainview Hospital, searching for the radiology department at Plainview Hospital will return all informed consents provided to that radiology department, and so on.

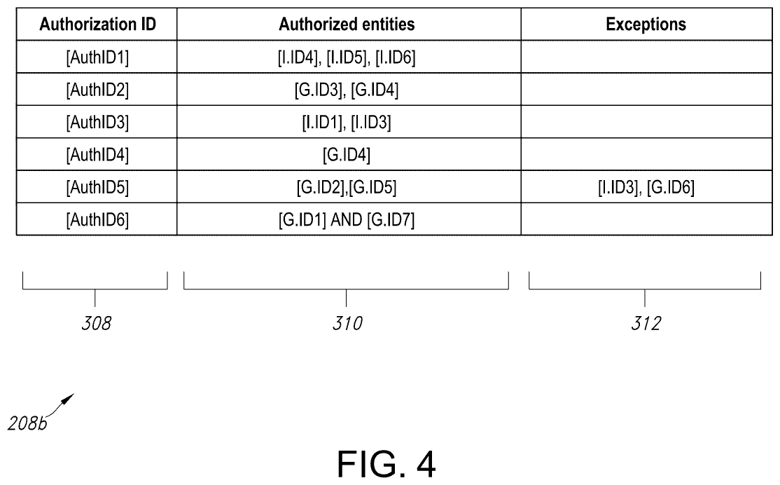

FIG. 4 depicts an example table 208b, which includes each patient's informed consents. In particular, the table 208b includes column 308, which includes authorization IDs, and column 310, which includes entities authorized to access the respective patient's confidential medical information (also referred to herein as a patient medical record). The example table 208b may optionally include a column 312, which may include exceptions to the corresponding authorized entities. For example, in table 208a (FIG. 3), Mr. Fleischmann's name corresponds to [AuthID5]. Table 208b shows [AuthID5] as corresponding to entities [G.ID2] and [G.ID5] (where “G” signifies “group”). This means that the entities associated with [G.ID2] and [G.ID5] have Mr. Fleischmann's informed consent to access Mr. Fleischmann's medical records. However, the entities in column 312 (e.g., [I.ID3] and [G.ID6]) are excepted, meaning that the entities in column 312 are expressly prohibited from accessing Mr. Fleischmann's confidential medical records. For example, [G.ID6] may be a sub-entity (department, group, etc.) of [G.ID5]. In some examples, the column 312 may include additional information regarding such exceptions at a greater level of granularity, such as the ability to view some records but not others; the ability to view records but not modify or distribute the records; the ability to distribute the records but not view the records; the ability to view some portions of records but not others; the ability to view non-restricted portions of records, and so on. More generally, the table 208b may include additional, relevant information for the various authorization IDs, such as the entities given informed consent (column 310), sub-entities given (column 310) or not given (column 312) informed consent, specific providers given (column 310) or not given (column 312) informed consent, the scope of the informed consent (e.g., whether and which specific parts of records may be viewed and under which conditions), and so on. For example, AuthID1 may be associated with entities [I.ID4], [I.ID5], and [I.ID6], meaning that these three entities are provided with informed consent. The table 208b may include any additional permissions associated with AuthID1, such as sub-entities within [I.ID4] to whom informed consent is granted and other such sub-entities from whom informed consent is withheld, the scope of any such granted or withheld informed consent (e.g., a sub-entity within [I.ID4] may access insurance information associated with AuthID1, but not genetic information associated with AuthID1). Other types of tables are also contemplated, such as a negative consent table in which specific authorization IDs are associated with withheld or refused informed consents. For example, entities or sub-entities associated with a given authorization ID may be refused access to specified records, specified aspects of specified records, and so on. Any and all such tables are accessible via the IMS 104 as described below. Further, informed consents may be searchable by any classification of datum stored in any of the tables described herein, for example using the search feature described below with reference to FIG. 13.

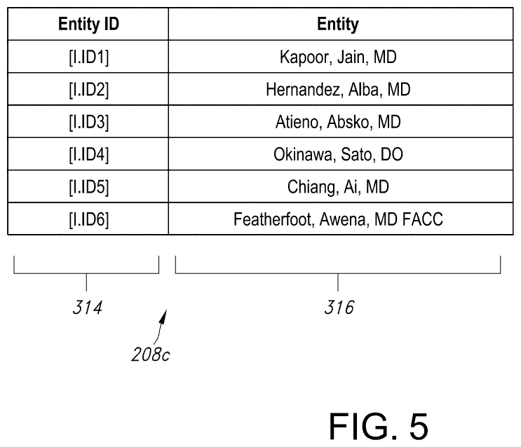

Table 208c in FIG. 5 enumerates the entities (e.g., individual persons) associated with entity IDs [I.ID1] through [I.ID6]. Column 314 includes entity IDs, and column 316 includes corresponding entity names. Thus, table 208c is useful to identify the entity [I.ID3] that Mr. Fleischmann indicated in his informed consent as an exception to whom his medical information may not be provided. Table 208c cross-references [I.ID3] with Absko Atieno, M.D., meaning that Mr. Fleischmann has prohibited Dr. Atieno from accessing Mr. Fleischmann's confidential medical information.

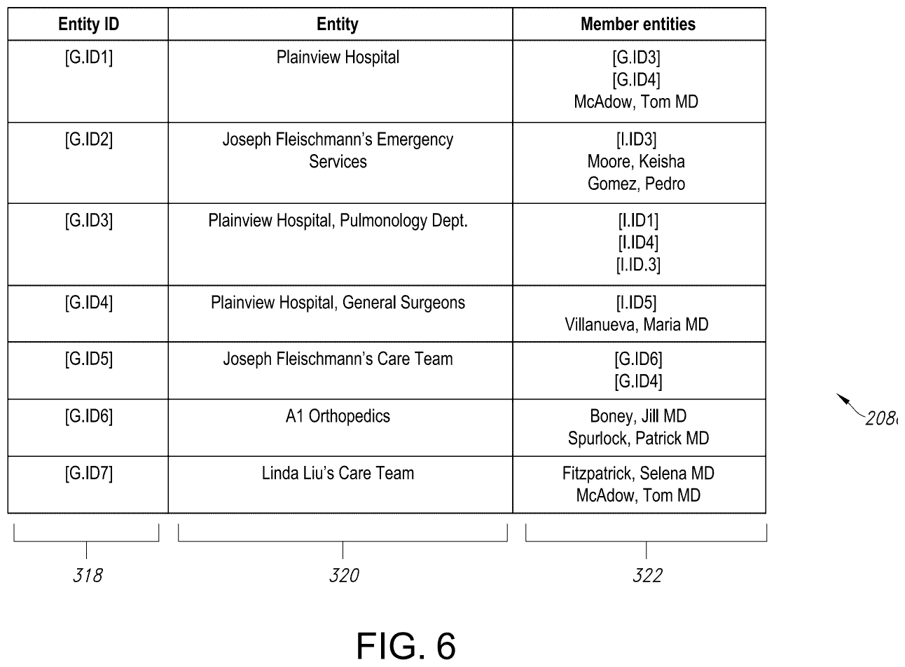

Table 208d in FIG. 6 enumerates the entities (e.g., groups) associated with entity IDs [G.ID1] through [G.ID7] (column 318). Column 320 provides the corresponding entity names. Column 322 enumerates the specific entities that are members of the corresponding entities indicated in column 320. For example, table 208b (FIG. 4) indicates that Joseph Fleischmann has provided consent to entities having entity IDs [G.ID2] and [G.ID5]. As table 208d (FIG. 6) shows, Entity ID [G.ID2] corresponds to Joseph Fleischmann's Emergency Services, a group that may be formed by an administrator of the IMS 104 (or by any other suitable person or device). Members of Joseph Fleischmann's Emergency Services group include Keisha Moore, Pedro Gomez, and the entity having entity ID [I.ID3]. Table 208c (FIG. 5) indicates that [I.ID3] is Dr. Atieno. Thus, because Mr. Fleischmann's [AuthID5] includes [G.ID2], Keisha Moore, Pedro Gomez, and Dr. Atieno are considered to have been granted informed consent to access Mr. Fleischmann's confidential medical information. Still referring to FIG. 6, [G.ID5] corresponds to Joseph Fleischmann's Care Team, which includes [G.ID6] and [G.ID4] as member entities. Entity ID [G.ID6] corresponds to the group A1 Orthopedics, which includes Jill Boney, M.D. and Patrick Spurlock, M.D. as member entities. Further, entity ID [G.ID4] corresponds to the group Plainview Hospital, General Surgeons and includes the entity corresponding to entity ID [I.ID5] and Maria Villanueva, M.D. as member entities. Table 208c indicates that entity ID [I.ID5] is Ai Chiang, M.D. Thus, tables 208b-208d indicate that Mr. Fleischmann has granted informed consent to Keisha Moore, Pedro Gomez, and Dr. Atieno, who are members of the Joseph Fleischmann's Emergency Services group, and also to Drs. Boney, Spurlock, Villanueva, and Chiang, who are members of the group Plainview Hospitals, General Surgeons and A1 Orthopedics groups, which in turn are members of Joseph Fleischmann's Care Team.

As described above, table 208b identifies [I.ID3] and [G.ID6] as exceptions to the informed consent Mr. Fleischmann provided to [G.ID2] and [G.ID5]. The entity ID [I.ID3] corresponds to Dr. Atieno, and the entity ID [G.ID6] corresponds to A1 Orthopedics. Consequently, although Mr. Fleischmann's consent is granted to all members of [G.ID2] (the group Joseph Fleischmann's Emergency Services), [I.ID3] (Dr. Atieno) is an exception, and Dr. Atieno is expressly prohibited from accessing Mr. Fleischmann's confidential medical information. Further, although Mr. Fleischmann's consent is granted to all members of [G.ID5] (the group Joseph Fleischmann's Care Team), [G.ID6] (A1 Orthopedics) is an exception, and thus all member entities of A1 Orthopedics are expressly prohibited from accessing Mr. Fleischmann's confidential medical information.

Referring to table 208d (FIG. 6), the member entities indicated in column 322 may be subject to change. For instance, as employees are hired and terminated, the member entities (column 322) of the entities (column 320) may be updated, such as by the administrator of the IMS 104 or by another suitable person. In examples, the member entities of column 322 may be updated automatically. More generally, any and all fields of the various tables 208a-208d may be updated, either by an administrator, another suitable person, or automatically. In examples, tables 208a-208d may be automatically updated by a syncing process with one or more databases (e.g., which may be stored internally or externally to the IMS 104), as a result of one or more persons leaving an organizational entity to which informed consent has been granted, as a result of a sub-entity no longer being associated with an entity to which informed consent has been granted, and so on. In examples, the patient may be provided the option to approve or deny such automatic updates, depending on the specific profile settings that the patient may have enabled. Because the member entities of each group are subject to change, the authorized entities indicated in column 310 of table 208b (FIG. 4) may, in some instances, specify groups, rather than individuals within those groups. Accordingly, as the member entities within the groups change over time, the corresponding informed consents will reflect changes to the groups. Similarly, in some examples, the member entities of each group may be stored in one or more databases external to the IMS 104. In such examples, the IMS 104 may be configured to obtain the most current indication of member entities for each group on a periodic basis, on a push basis (e.g., whenever there are updates to the member entities for a group), and/or whenever the IMS 104 is queried for a particular patient's informed consents.

The examples described herein assume that the IMS 104 stores patient consents in the tables 208 in memory 202. However, in examples, the tables 208 may be stored in another device that is accessible to the IMS 104, such as a server in a different geographical location with which the IMS 104 is able to communicate. On a periodic basis, on a push basis, or when queried, the IMS 104 may access these tables 208 to obtain and provide accurate consent information.

Patient location may be useful in updating informed consents. For example, the location of the patient device 102 (FIG. 1) may be transmitted to IMS 104) when IMS 104 is accessed, or periodically (e.g., based on a patient profile or settings associated with a dynamic informed consent management application on patient device 102). Upon determining that the patient device 102 is in a specific location or detecting a location change (e.g., change of state, country, or jurisdiction), IMS 104 may prompt the patient using the patient device 102 to dynamically update or otherwise manage the patient's informed consent, such as to update entities and/or sub-entities to whom informed consent is granted or from whom informed consent is withheld, the types of consent granted or withheld, etc. For example, upon determining that the patient device 102 is in a hospital, the IMS 104 may prompt the patient to dynamically manage informed consents, as entry to a hospital may be an opportune time to update informed consent.

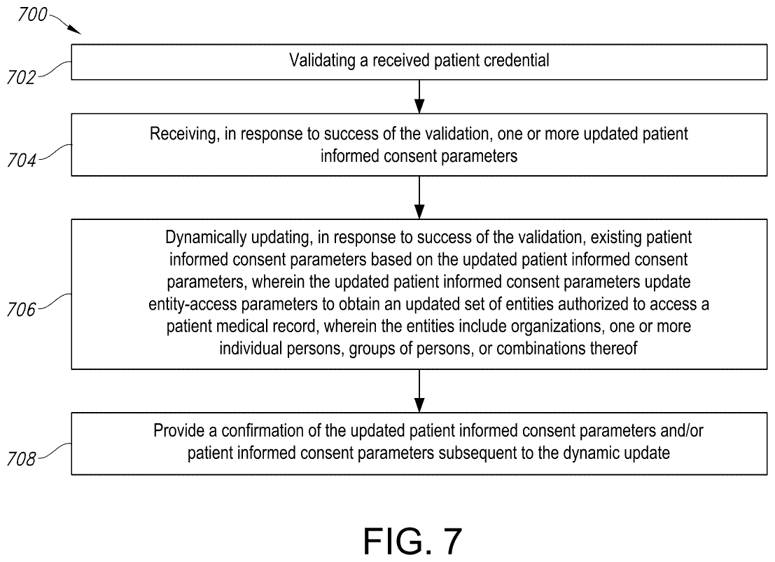

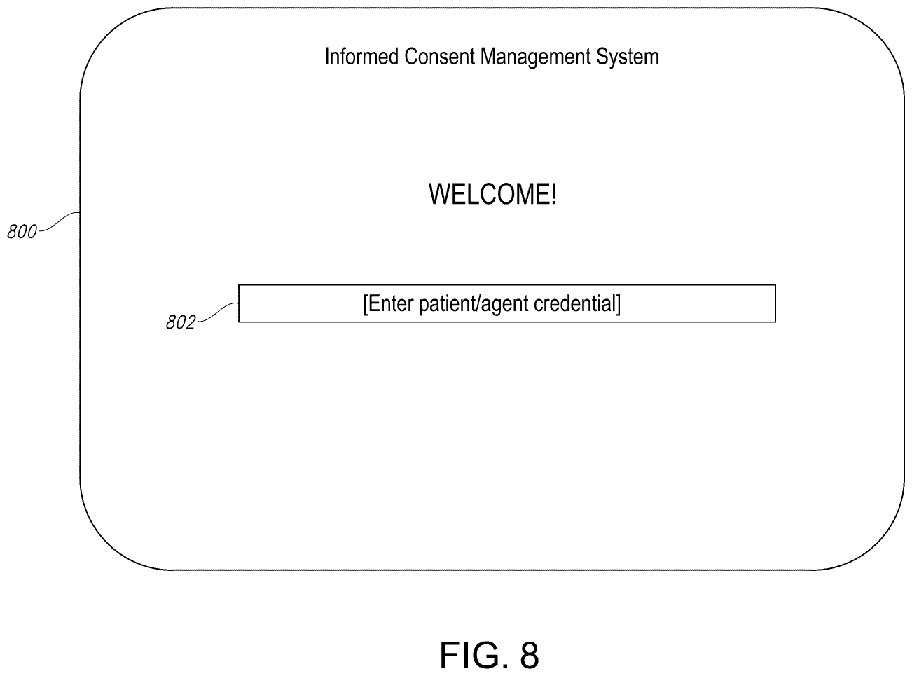

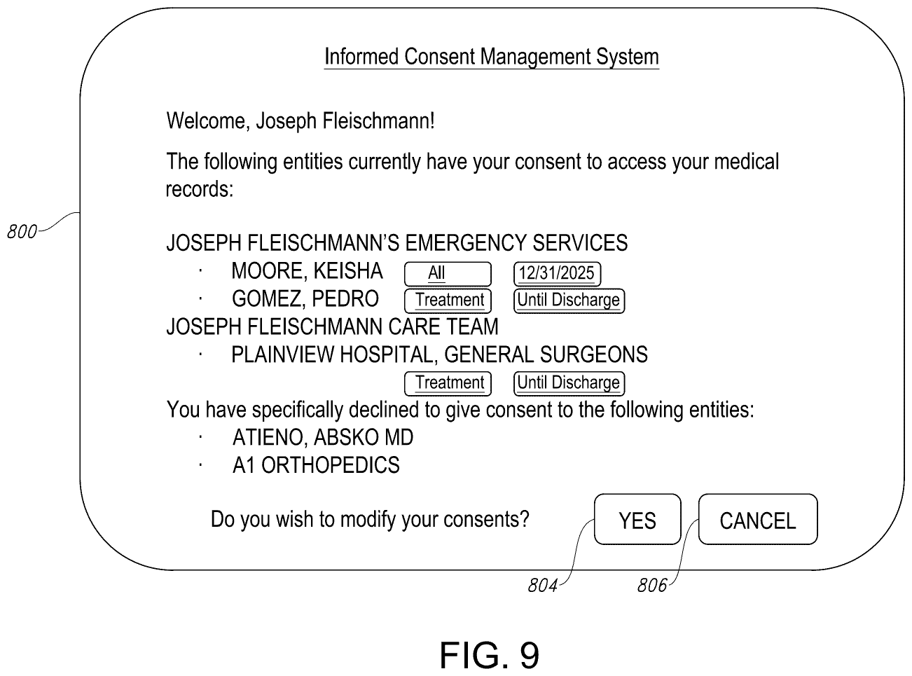

FIG. 7 is a flow diagram of a method 700 for dynamically managing informed consents, in accordance with various examples. The steps of the method 700 may be performed by any suitable device. In examples, the IMS 104 (FIGS. 1 and 2) may performs method 700, although the scope of this disclosure is not limited as such. The method 700 includes validating a received patient credential provided by a device (702). For instance, continuing with the above examples, the patient Mr. Fleischmann may access a GUI 800 (FIG. 8) on a patient device 102 (FIG. 1), such as a mobile device, and enter his credential in field 802 (FIG. 8). Upon entering the appropriate credential (in Mr. Fleischmann's case, [Credential5a] (FIG. 3)) into field 802, the patient device 102 provides the credential to the IMS 104. In turn, the IMS 104 validates the credential and grants the patient device 102 access to Mr. Fleischmann's consents. In some embodiments, as FIG. 9 shows, logging in with the appropriate credential may cause the GUI 800 to display Mr. Fleischmann's current consents and exceptions to those consents by entity/sub-entity. Additional information may be included, such as a description of the consent provided (e.g., “all” or “treatment” as shown) and the date at which the consent expires (e.g., “Dec. 31, 2025” or “until discharge” as shown). Mr. Fleischmann may be provided the opportunity to dynamically customize his informed consents by tapping a button 804, or Mr. Fleischmann may end the session by tapping a button 806. The GUI 800 shown in FIGS. 8 and 9 is merely an example. The GUI 800 may also include options to select and view entity privileges based on specific parameters, granularity levels, etc.

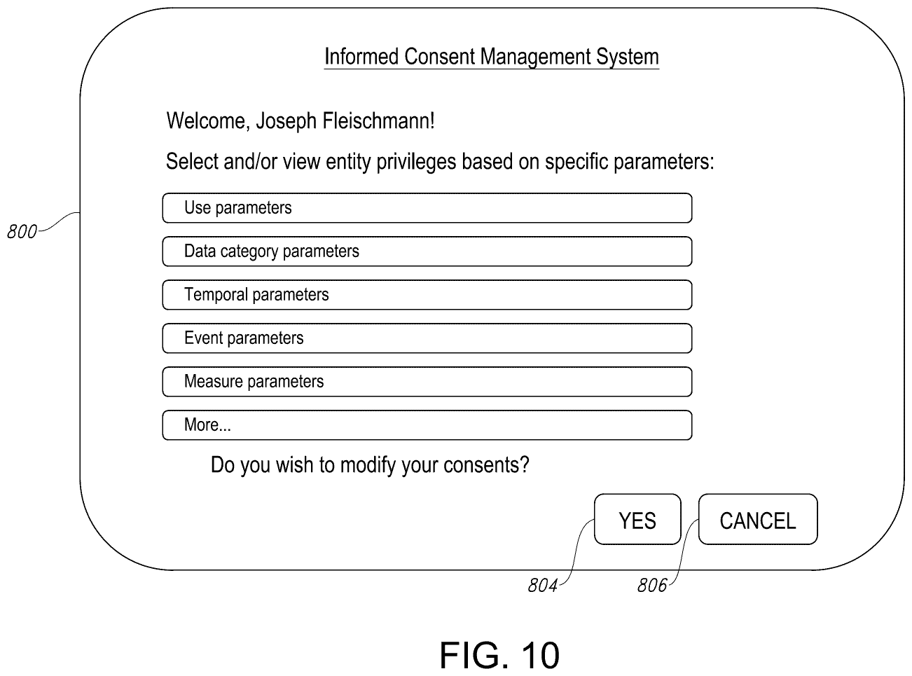

As FIG. 10 shows, Mr. Fleischmann may select and/or view entity privileges based on such specific parameters, examples of which may include: use parameters governing access by one or more entities in the updated set of entities to the patient medical record; data-category parameters governing access to sections of the patient medical record by the one or more entities in the updated set of entities; or temporal parameters determining a duration of access by the one or more entities in the updated set of entities to the patient medical record; or event parameters associating access to the patient medical record by the one or more entities in the updated set of entities to the occurrence or non-occurrence of specific events; or measure parameters associating use of the patient medical record by the one or more entities in the updated set of entities to one or more measures; or a combination thereof. More particularly, at a first level of use granularity, use parameters enable use of the patient medical record for one or more specified uses and prevent use of the patient medical record for uses other than the one or more specified uses. Examples may include permitting or denying use of clinical data for diagnosis and/or treatment and/or follow ups, and/or clinical trials, and/or research, and/or billing, and/or insurance, etc., all or any of which may be provided on a per-entity or per-sub-entity basis. As an example, access to and use of the patient medical record may be allowed for treatment and billing but denied for use in research including genetic research. At a first level of data category granularity, the data category parameters enable access to a first section of the patient medical record and prevent access to a second section of the patient medical record, where the first section and the second section represent distinct data categories. Data category granularity may allow access to one portion of a patient medical record, such as a clinical record for diagnosis of a specific condition, but prevent access to another portion of the patient medical record, such as a psychiatric record. At a first level of temporal granularity, the temporal parameters enable access to the patient medical record for a specified period. At a first level of event granularity, the event parameters enable the access to the patient medical record based on an event, where the events comprise at least one of: an emergency event, a medical procedure event, a treatment related event, a diagnostic event, a post-treatment event, a billing event, or a clinical trial event, or a research event, or a combination thereof. At a first level of measure granularity, the measure parameters enable the access or use of the patient medical record based on measures associated with the patient medical record, where the measures comprise at least one of: de-identification, aggregation, anonymization, or pseudo-anonymization. In embodiments, the measure parameters are implicitly determined based on a corresponding jurisdiction associated with the one or more entities in the updated set of entities.

The current consents and exceptions to those consents are obtained using tables 208a-208d, as described in detail above, and are displayed to Mr. Fleischmann on the GUI 800. Mr. Fleischmann may be provided the opportunity to dynamically customize his informed consents by tapping the button 804, or Mr. Fleischmann may end the session by tapping the button 806. Similarly, in some embodiments, as FIG. 11 shows, logging in with the appropriate credential may cause the GUI 800 to display buttons or other GUI objects (such as icons, pull down menus, etc.) that can be selected to display entities based on type of information. For instance, and as shown, Mr. Fleischmann may select a button that will result in the display of all entities that have access to Mr. Flesichmann's genetic information, or a button that will result in the display of all entities that have access to Mr. Fleischmann's medical conditions, or a button that will result in the display of all entities that have access to a record of Mr. Fleischmann's medications. Records of such entities also may be obtained through a search query, as shown. Likewise, in some embodiments, as FIG. 12 shows, logging in with the appropriate credential may cause the GUI 800 to display buttons that can be selected to display entities based on a relevant use case. For instance, and as shown, Mr. Fleischmann may select a button that will result in the display of all entities that have research privileges to access Mr. Fleischmann's medical records, or a button that will result in the display of all entities that have access to a record of Mr. Fleischmann's medical records because they are part of an authorized clinical care team, or a button that will result in the display of all entities that have access to a record of Mr. Flesichmann's medical records by law or by agreement (e.g., power of attorney). Records of such entities also may be obtained through a search query, as shown.

Responsive to tapping button 804, the patient device 102 shows Mr. Fleischmann an updated GUI 800, for example as illustrated in FIG. 13. To add entities to Mr. Fleischmann's informed consents, Mr. Fleischmann may tap the drop down menu 808. To remove entities from Mr. Fleischmann's informed consents, Mr. Fleischmann may tap the drop down menu 810. To specify exceptions to Mr. Fleischmann's informed consents, Mr. Fleischmann may tap the drop down menu 812. To save changes, Mr. Fleischmann may tap the SAVE button 814. To manage Mr. Fleischmann's agents' (e.g., next-of-kin, or those with medical power of attorney) credentials, and thus the agents' ability to manage Mr. Fleischmann's consents, Mr. Fleischmann may tap the MODIFY AGENT button 816. The button 816 may be available to patients logged in under Mr. Fleischmann's credential and not to those logged in under an agent's credential. Mr. Fleischmann may tap the LOG OUT button 817 to end the session.

The method 700 includes receiving, responsive to success of the validation, one or more updated patient informed consent parameters (704). The one or more updated patient informed consent parameters may be provided by a person, such as Mr. Fleischmann, through the GUI 800. Examples of such parameters may include those described above, such as the use parameters governing access by one or more entities in the updated set of entities to the patient medical record; data category parameters governing access to sections of the patient medical record by the one or more entities in the updated set of entities; temporal parameters determining a duration of access by the one or more entities in the updated set of entities to the patient medical record; event parameters associating access to the patient medical record by the one or more entities in the updated set of entities to the occurrence or non-occurrence of specific events; or measure parameters associating use of the patient medical record by the one or more entities in the updated set of entities to one or more measures; or a combination thereof.

A patient, such as Mr. Fleischmann, may dynamically manage his or her informed consent in a variety of ways using the GUI 800 of FIG. 13. In a first example, the patient may provide informed consent to individuals, revoke consent from individuals, and/or manage consent exceptions on an individual basis. For instance, referring to tables 208a and 208b (FIGS. 3 and 4), patient John Williams may log in with his patient device 102 and customize his [AuthID1] to include authorized entities [I.ID4], [I.ID5], and [I.ID6], all of which are individual persons. According to table 208c (FIG. 5), these entities are Drs. Sato Okinawa, Ai Chiang, and Awena Featherfoot, respectively.

In a second example, the patient may provide informed consent to groups, revoke consent from groups, and/or manage consent exceptions on a group basis. For instance, referring to tables 208a and 208b (FIGS. 3 and 4), patient Matthew Kennedy may log in with his patient device 102 and customize his [AuthID2] to include authorized entities [G.ID3] and [G.ID4], each of which is a group. According to table 208d (FIG. 6), the former is Plainview Hospital, Pulmonology Department, and the latter is Plainview Hospital, General Surgeons. In this manner, Mr. Kennedy has provided informed consent to all member entities of the pulmonology and general surgery groups at Plainview Hospital. Managing informed consent given to or withheld from a particular entity (e.g., group) may also include managing informed consent given to or withheld from sub-entities within the group. Such management may include the type of consent, scope of consent, duration of consent, and so on.

In a third example, the patient may provide informed consent to, revoke consent from, and/or manage consent exceptions for those entities that are present in multiple groups. For instance, referring to tables 208a and 208b (FIGS. 3 and 4), patient Linda Liu may log in with her patient device 102 and customize her [AuthID6] to include authorized entities [G.ID1] and [G.ID7], each of which is a group. In this case, however, Ms. Liu has made appropriate selections on the GUI 800 that effectively place an AND Boolean operator linking the entities [G.ID1] and [G.ID7], meaning that those entities who are present in both groups [G.ID1] and [G.ID7] are granted consent. Referring to table 208d (FIG. 6), the overlap between groups [G.ID1] and [G.ID7] is Tom McAdow, M.D., and thus Dr. McAdow is provided consent to access Ms. Liu's confidential medical information.

In a fourth example, the patient may provide informed consent to, revoke consent from, and/or manage consent exceptions for a group irrespective of that group's relationship to any other group. For instance, Ms. Liu may consent to giving her confidential medical information to [G.ID7], which is Linda Liu's Care Team. The member entities for this team, according to table 208d (FIG. 6), are Drs. Fitzpatrick and McAdow. Dr. McAdow belongs to [G.ID1] (the Plainview Hospital group), and Dr. Fitzpatrick does not, but unlike the third example above, in this fourth example, Ms. Liu's consent is still given to all member entities of [G.ID7], Linda Liu's Care Team, irrespective of whether and to what degree that group overlaps in membership with any other group.

In a fifth example, the patient may provide informed consent to, revoke consent from, and/or manage consent exceptions for an emergency services group that is treating the patient. For example, if patient Mr. Fleischmann is riding in an ambulance to the hospital, Mr. Fleischmann may use the patient device 102 to override any existing consents or exclusions to provide immediate access to any and all member entities of [G.ID2], the Joseph Fleischmann's Emergency Services group. In examples, such an emergency services override feature may be available for the patient to select under drop down menu 808 on GUI 800 (FIG. 13). The emergency services group for any particular patient may be populated and made available to the patient by the administrator for the IMS 104 or by any other suitable entity. In the event the patient is unconscious or otherwise unable to make competent medical decisions, the patient's agent may be able to select the emergency services override feature.

In a sixth example, the patient may specify exceptions to consents, as described in detail above. Exceptions may be valid as long as the consents are valid.

In a seventh example, the patient may provide informed consent to, revoke consent from, and/or manage consent exceptions for select departments or specialties within certain groups. For example, the patient may provide consent to a cardiology group within a hospital, a nephrology group within a hospital, a radiology group within a clinic, emergency medical technicians (EMTs) in an ambulance service, nurses in an intensive care unit (ICU) of a hospital, and so on. For instance, according to tables 208a-208d (FIGS. 3-6), Mr. Kennedy has provided consent to entities [G.ID3] and [G.ID4], which are the pulmonology and general surgery departments within Plainview Hospital, respectively.

In an eighth example, and as described above, a patient may designate one or more agents who can provide informed consent to, revoke consent from, and/or manage consent exceptions for entities on the patient's behalf. Each such agent may be assigned a separate login credential, so that the patient's login credential has adequate clearance to manage the authorized agent(s). Referring to table 208a (FIG. 3), Messrs. Kennedy and Fleischmann have specified agents, while Messrs. Williams and Harris and Mss. Feinstein and Liu have not.

In a ninth example, a patient may dynamically manage the type of informed consents provided to a particular entity or sub-entity. For example, a patient may change the nature of consent provided to each entity or sub-entity, such as by altering the designation of informed consent between various categories such as clinical, research, billing, insurance, etc. Thus, for instance, a physician (e.g., Dr. Jain Kapoor in FIG. 5) may be granted a clinical level of informed consent, with all permissions and restrictions that accompany the clinical level of informed consent, but the patient may subsequently modify the informed consent from the clinical level to the researcher level, with all permissions and restrictions that accompany the researcher level of informed consent. The patient may adjust patient settings (e.g., by way of the GUI 800) to describe the specific permissions and restrictions associated with each such level.

In a tenth example, a patient may dynamically modify the type of data to which the informed consent parameters apply for each entity or sub-entity. For example, one of the tables 208a-208c may be modified to specify that particular informed consents apply to particular types of data, such as genetic data, procedure data, diagnostic data, imaging data, and so on. For instance, a patient may specify that Dr. Alba Hernandez (FIG. 5) is given informed consent with unencumbered access to all of the patient's genetic testing data but not procedure-related data. Thus, Dr. Hernandez may be unaware of the patient's specific surgical history. However, the patient may use the GUI 800 to dynamically modify the informed consent given to Dr. Hernandez to include the patient's procedure-related data.

In an eleventh example, the management of informed consents may be performed automatically, on a patient's behalf, subject to approval by the patient. Such management may be performed, for example, by the IMS 104 or by any other suitable device. Such management may include repeatedly modifying informed consents based on a workflow approach to the healthcare experience, in which a first entity (e.g., first responder, such as a paramedic) is provided with unencumbered informed consent to all patient records. Subsequently, a physician at a hospital to which the patient is taken may be provided with unencumbered informed consent to all patient records, while the paramedic informed consent is revoked. During the inpatient stay, informed consent may be provided to and/or withheld from any of a variety of healthcare professionals, including hospitalists, surgeons, nurses, laboratory personnel, phlebotomists, and so on. After treatment has been completed, informed consent may be revoked for all such personnel and may be provided to administrative, billing, and insurance staff at the hospital. After accounts have been settled, informed consent may be revoked from all hospital employees. Each step in this process may be performed automatically, with the patient receiving prompts on the patient device 102 to provide authorization for each modification to the patient's informed consents. Each such prompt may include proposed levels of records access to each user, which the patient can dynamically modify, such as by selecting or unselecting radio buttons provided on the GUI 800. Similarly, each such prompt may include proposed durations of access (e.g., either by discrete units of time, or by phases of the hospital stay, or until further notice), each of which the patient may dynamically modify. The IMS 104 may provide informed consent proposals for specific users based on database directories for relevant entities. For instance, the location of the patient device 102 may trigger the IMS 104 to determine that the patient has been admitted to a hospital at which the patient device 102 is located, and based on an available directory of that hospital, the IMS 104 may provide the patient device 102 with the various options described above. Other options may be provided as well in addition to informed consent modifications, such as legal documents for do not revive/resuscitate orders, modifications and/or exceptions to proposed informed consents, and so on.

The eleventh example described above is a form of use-specific authorization, in which a patient grants informed consent to an entity for a specific use and for a finite duration. In the above eleventh example, the patient grants informed consent to the paramedic for a specific instance (e.g., the injury sustained on the date of service) and for a specific duration (e.g., until arrival at the hospital). Use-specific authorizations are not limited to the automated informed consent example described above and may also be used in any of a variety of contexts. For example, a patient may visit an urgent care clinic while traveling on business and may grant informed consent to the urgent care clinic and/or providers, administrative or billing staff, etc. within the urgent care clinic for the duration of the visit. The informed consent may be revoked for some or all of the entities associated with the urgent care clinic after service has been completed. For instance, informed consent may be revoked for the physicians at the urgent care clinic after the patient leaves the clinic, but billing staff at the urgent care clinic may retain informed consent until the patient account has been settled.

In a twelfth example, a patient may provide informed consent to a user based on specific conditions that are to be met. For example, the patient may specify that a specific family physician is authorized to access the patient's medical record when one or more conditions (e.g., a specific time period since the most recent clinic visit) is satisfied. Similarly, the patient may grant universal informed consent to all entities meeting the conditions (e.g., entities in a certain geographical location, and the current date being within a specific time period since the most recent clinic visit).

As shown in FIG. 13, the GUI 800 may include a search query field 818. The search query field 818 may be populated by a patient or other user of the GUI 800 with one or more search terms that may be captured by a processor, such as processor 200 (FIG. 2). The processor 200 may use the captured search term(s) to search the tables 208, the memory 202, and/or any other information, whether inside or outside the IMS 104. The search query field 818 may be useful to search by one or more parameters. For example, the search query field 818 may be useful for the patient to search for all entities to whom informed consent has been given. The search query field 818 may be useful to search for all entities from whom informed consent has been withheld. The search query field 818 may be useful to search for particular types of entities to whom informed consent has been given or from whom informed consent has been withheld, such as billing entities, administrators, researchers, medical professionals, and so on. The search query field 818 may be useful to search for entities satisfying multiple such criteria. For example, the patient may search for entities to whom consent has been given or from whom consent has been withheld and who also satisfy multiple criteria, such as medical professionals who are cardiologists, billing entities who work with gastroenterologists, administrators in hospitals that qualify as level 1 trauma centers, and so on. The patient is able to use the search query field 818 to construct queries of any complexity level, using Boolean or other types of logic.

Referring to FIG. 7, the method 700 includes dynamically updating, in response to success of the validation, existing patient informed consent parameters based on the updated patient informed consent parameters, where the updated patient informed consent parameters update entity-access parameters to obtain an updated set of entities authorized to access a patient medical record, and where the entities include organizations, one or more individual persons, groups of persons, or combinations thereof (706). In some embodiments, the step 706 may include using the updated patient informed consent parameters provided by Mr. Fleischmann via the GUI 800 (e.g., changes to entities that are authorized to access Mr. Fleischmann's medical records and those that are not authorized to access Mr. Fleischmann's medical records) to update existing patient informed consent parameters that are stored in various tables, such as any of the tables 208a-208d described in detail above.

In some embodiments, in block 708, a confirmation of the updated patient informed consent parameters (e.g., as updated by the patient) and/or patient informed consent parameters subsequent to the dynamic update (e.g., as existing subsequent to the update) may be provided. In some embodiments, the confirmation or indication of confirmation may be provided in the form of a summary of changes, a report, or in another form (e.g., as configured by the patient and/or based on the patient profile and/or based on the nature of the updates).

The previous description of the disclosed examples is provided to enable any person skilled in the art to make or use the features described in the present disclosure. Various modifications to these examples will be readily apparent to those skilled in the art, and the generic principles defined herein may be applied to other examples without departing from the scope of the disclosure. The present disclosure is not intended to be limited to the examples shown herein but is to be accorded the widest scope consistent with the principles and novel features disclosed herein.

The following is claimed:

1. A processor implemented method, comprising:
validating a received patient credential;
receiving, in response to success of the validation, one or more updated patient informed consent parameters; and
dynamically updating, in response to success of the validation, existing patient informed consent parameters based on the updated patient informed consent parameters, wherein the updated patient informed consent parameters update: entity-access parameters to obtain an updated set of entities authorized to access a patient medical record, wherein the entities include organizations, one or more individual persons, groups of persons, or combinations thereof.

2. The method of claim 1, wherein the updated patient informed consent parameters further update at least one of:
use parameters governing access by one or more entities in the updated set of entities to the patient medical record; or
data category parameters governing access to sections of the patient medical record by the one or more entities in the updated set of entities; or
temporal parameters determining a duration of access by the one or more entities in the updated set of entities to the patient medical record; or
event parameters associating access to the patient medical record by the one or more entities in the updated set of entities to the occurrence or non-occurrence of specific events; or
measure parameters associating use of the patient medical record by the one or more entities in the updated set of entities to one or more measures; or
a combination thereof.

3. The method of claim 2, wherein receiving the one or more updated patient informed consent parameters comprises receiving the updated patient informed consent parameters at differing levels of granularity.

4. The method of claim 3, wherein,
at a first level of entity-access granularity, the updated patient informed consent parameters enable a first entity to access the patient medical record by including the first entity in the updated set of entities, and revoke access to the patient medical record to a second entity by excluding the second entity from the updated set of entities and wherein,
at a second level of entity-access granularity, patient authorization enables
a first sub-entity associated with the first entity to access the patient medical record, or
prevents a second sub-entity associated with the first entity from accessing the patient medical record, or
a combination thereof.

5. The method of claim 3, wherein, at a first level of use granularity, use parameters enable use of the patient medical record for one or more first specified uses and prevents use of the patient medical record for second uses other than the one or more first specified uses.

6. The method of claim 3, wherein, at a first level of data-category granularity, the data category parameters enable access to a first section of the patient medical record and prevent access to a second section of the patient medical record, wherein the first section and the second section represent distinct data categories.

7. The method of claim 3, wherein at a first level of temporal granularity the temporal parameters enable access to the patient medical record for a specified period.

8. The method of claim 3, wherein, at a first level of event granularity, the event parameters enable the access to the patient medical record based on an event, wherein the events comprise at least one of: an emergency event, a medical procedure event, a treatment related event, a diagnostic event, a post-treatment event, a billing event, or a clinical trial event, or a research event, or a combination thereof.

9. The method of claim 3, wherein at a first level of measure granularity, the measure parameters enable the access or use of the patient medical record based on measures associated with the patient medical record, wherein the measures comprise at least one of: de-identification, aggregation, anonymization, or pseudo-anonymization.

10. The method of claim 9, wherein the measure parameters are implicitly determined based on a corresponding jurisdiction associated with the one or more entities in the updated set of entities.

11. A system, comprising a memory, a communications interface, and a processor coupled to the communications interface, wherein the processor is configured to:
receive and validate a patient credential of a patient;
in response to success of the validation, receive one or more updated patient informed consent parameters; and
dynamically update, in response to success of the validation, existing patient informed consent parameters based on the updated patient informed consent parameters, wherein the updated patient informed consent parameters update: entity-access parameters to obtain an updated set of entities authorized to access a patient medical record, wherein the entities include organizations, one or more individual persons, groups of persons, or combinations thereof.

12. The system of claim 11, wherein the processor is configured, after validating the received patient credential and before receiving the one or more updated patient informed consent parameters, to provide a graphical user interface (GUI) to a patient, the GUI configured to receive the updated patient informed consent parameters at differing levels of granularity.

13. The system of claim 12, wherein, at a first level of granularity, the GUI is configured to enable the patient to authorize an entity to access the patient medical record, and wherein, at a second level of granularity, the GUI is configured to enable the patient to authorize a first sub-entity belonging to the entity to access the patient medical record and to prevent a second sub-entity belonging to the entity from accessing the patient medical record.

14. The system of claim 12, wherein, at a third level of granularity, the GUI enables the patient to authorize an entity to access a first portion of the patient medical record and not a second portion of the patient medical record.

15. The system of claim 12, wherein the GUI enables the patient to authorize an entity to access the patient medical record for a finite period of time.

16. The system of claim 12, wherein the GUI enables the patient to specify conditions under which an entity is authorized to access the patient medical record.

17. The system of claim 12, wherein the GUI enables searching of the existing informed consent parameters.

18. The system of claim 17, wherein the GUI enables searching of the existing informed consent parameters by entity, sub-entity, medical provider, scope of consent, type of consent, and nature of consent.

19. The system of claim 17, wherein the GUI is configured to display entities according to the nature of consent granted.

20. A non-transitory, computer-readable medium storing executable instructions which, when executed by a processor, cause the processor to:
validate a received patient credential;
receive, in response to success of the validation, one or more updated patient informed consent parameters; and
dynamically update, in response to success of the validation, existing patient informed consent parameters based on the updated patient informed consent parameters, wherein the updated patient informed consent parameters update at least one of: a set of entities authorized to access a patient medical record, entity-specific access authorizations to specific portions of the patient medical record, and use-specific authorizations, the entities including one or more individual persons, groups of persons, or combinations thereof.
